(19) United States
(12) Patent Application Publication
(10) Pub. No.: US 2026/0315281 A1
(43) Pub. Date: Oct. 15, 2026

(54) EYEWEAR WITH TEAR-OFF LENS GUARD SYSTEM

(71) Applicant: HEATWAVE VISUAL, Gilroy, CA (US)

(72) Inventor: Justin Marc HERTEL, Gilroy, CA (US)

(21) Appl. No.: 19/174,656

(22) Filed: Apr. 9, 2025

(51) Int. Cl. A61F 9/02; G02C 13/00

(52) U.S. Cl. CPC A61F 9/025; G02C 13/001

(57) ABSTRACT

A system for applying and removing protective films from eyewear includes a jig assembly with positioning pins, an eyewear with a curved lens, and a protective film with alignment features. The jig assembly includes an alignment rim matching the eyewear contour and removable stand elements that insert into receptacles to elevate the assembly. The protective film incorporates alignment features corresponding to the jig's positioning pins and a segmented release liner system enabling staged application. The film is applied by positioning the eyewear in the jig, aligning the film using the positioning pins, and sequentially removing release liner sections. Film removal is accomplished by bending the lens opposite to its natural curvature, causing the film to separate from the lens surface. The system provides precise film application while maintaining lens protection and enabling clean removal without residual adhesive.

### TECHNICAL FIELD

The present disclosure generally relates to protective eyewear systems and methods for maintaining optical clarity of eyewear. More specifically, the disclosure relates to eyewear protection systems, including protective films, application methods, and associated tools and fixtures for applying protective elements to curved lens surfaces. The disclosure further relates to frameless eyewear configurations and mechanisms for protecting and maintaining visibility of curved lens surfaces in various use environments.

### BACKGROUND

Eyewear has become an essential part of daily life, serving both protective and aesthetic purposes across many industries and activities. From sports and recreational activities to industrial and professional settings, protective eyewear plays a crucial role in safeguarding users' vision while maintaining optical clarity and comfort.

Traditional protective eyewear often faces challenges related to lens durability, visibility maintenance, and practical usability. When protective lenses become scratched, damaged, or obscured by debris, the entire eyewear unit typically needs replacement, leading to increased costs and waste. Additionally, conventional protective films or covers for eyewear lenses can be difficult to apply properly, often resulting in misalignment, air bubbles, or improper adhesion.

The process of applying protective films to curved lens surfaces presents particular challenges, as traditional application methods may not account for the complex geometries and varying curvatures found in modern eyewear designs. Furthermore, existing solutions often lack user-friendly features that would enable consistent and precise application of protective elements.

The field of protective eyewear continues to evolve, with ongoing efforts to develop more efficient and practical solutions for lens protection and maintenance. There remains a need in the industry for improved systems and methods that address these longstanding challenges while enhancing user experience and product longevity.

### BRIEF SUMMARY

In various embodiments, a system may include eyewear with a lens, wherein the lens is to be coated with a film. This film comprises an adhesive backing, a release liner, and one or more alignment features. Additionally, the system may incorporate a jig assembly, which includes one or more positioning pins that align with the alignment features of the film. The jig assembly is designed to securely hold the lens in a fixed position during the application process.

In some embodiments, the film comprises one or more alignment features, such as alignment pins, alignment tabs, or alignment markers.

In some embodiments, the jig assembly includes one or more positioning pins, which correspond to the alignment features of the film. The jig assembly is configured to retain the lens in a fixed position, ensuring accurate alignment and proper application of the film.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a diagram of a jig assembly, in accordance with some examples.

FIG. 2 is a diagram of a jig assembly, in accordance with some examples.

FIG. 3 is a diagram depicting eyewear being positioned onto a jig assembly, in accordance with some examples.

FIG. 4 is a diagram depicting eyewear secured in position on a jig assembly, in accordance with some examples.

FIG. 5 is a diagram depicting a protective film, in accordance with some examples.

FIG. 6 is a diagram depicting a protective film being applied to eyewear, in accordance with some examples.

FIG. 7 is a diagram depicting removal of a protective film from eyewear, in accordance with some examples.

### DETAILED DESCRIPTION

Protective eyewear systems face several critical challenges in maintaining optical clarity and functionality. When protective lenses become scratched, damaged, or obscured by debris, users typically must replace the entire eyewear unit, resulting in increased costs and unnecessary waste. The process of applying protective films to curved lens surfaces presents particular difficulties, as traditional application methods often result in misalignment, air bubbles, or improper adhesion due to the complex geometries and varying curvatures found in modern eyewear designs.

According to certain examples, the present system comprises specialized eyewear with a curved lens design, a protective film system, and a precision application jig assembly. The eyewear features a lens that curves along a single axis and includes fusion-bonded temples extending from opposite sides. A reinforcement rib positioned along the inside upper edge of the lens's backside provides structural stability and maintains the lens shape during use.

In certain examples, the lens may include a specialized surface treatment to optimize interaction with the protective film system. For example, these treatments may include a multi-layer, reflective mirror coating applied to the lenses (i.e., Revo coating or plasma treatment), which modify the surface tension characteristics and improve an interface between the lens and the adhesive backing of the protective film. The coating prevents residual adhesive from remaining on the lens surface after film removal, maintaining optical clarity between applications.

In certain examples, the protective film incorporates an adhesive backing and a segmented release liner system. The film includes alignment features that correspond with positioning pins on the jig assembly. For example, the positioning pins may be positioned along a common axis. The release liner comprises multiple independently removable sections: a center section containing the alignment features and side sections with removal tabs that enable controlled application. In some examples, the lens may include specialized surface treatments such as Revo coating or plasma treatment that enhance the interaction between the lens surface and the adhesive backing of the protective film. These specialized coatings create an interface that facilitates both secure adhesion during use and clean separation during removal. For example, the surface treatment may modify the surface tension characteristics of the lens, allowing the protective film to maintain proper adhesion while curved along a single axis, yet separate cleanly when the lens is flexed in the opposite direction.

The jig assembly serves as an application platform and may include one or more positioning pins that correspond with the film's alignment features. In some examples, the assembly may feature an alignment rim along its peripheral edge with a contour matching the eyewear shape and slots for engaging the eyewear temples. The jig may include a plurality of cylindrical receptacles formed in its bottom side for receiving removable stand elements that elevate the assembly during film application. When not in use, the stand elements can be secured by retention mechanisms on the bottom side of the jig.

For film application, the eyewear is positioned onto the jig assembly with its temples engaged in the alignment rim slots. The center section of the release liner is removed first, allowing alignment of the film's features with the jig's positioning pins. The side sections of the release liner are then sequentially removed to complete adhesion. Film removal is accomplished by bending the lens in a direction opposite to its natural curvature, causing the protective film to separate cleanly from the lens surface.

The jig assembly includes provisions for compact storage and organization. The stand elements can be removed and stored in dedicated receptacles positioned adjacent to storage positions on the jig's bottom surface. In some examples, the assembly may serve as a storage solution for the eyewear, with the temples folding inward to create a secure, compact unit that prevents loss and maintains protection of the components.

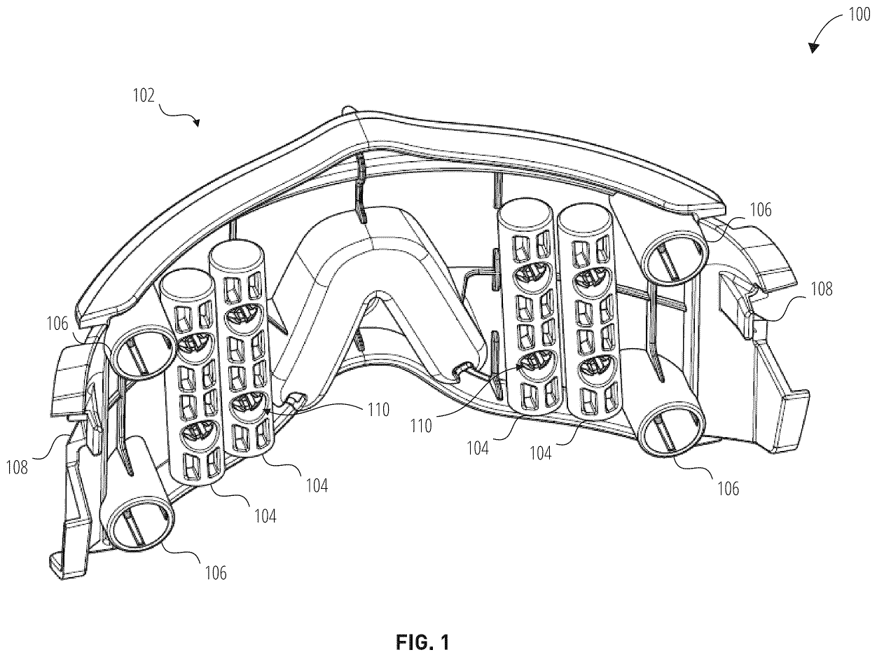

FIG. 1 is a diagram 100 of a jig assembly 102, in accordance with some examples, for applying protective films to eyewear. According to certain examples, the jig assembly 102 includes multiple positioning pins arranged on its front face, which are configured to align with corresponding features on the protective film.

In some examples, the jig assembly 102 includes a plurality (e.g., four) stand elements 104 and corresponding cylindrical receptacles 106 formed in the bottom side of the assembly. The receptacles 106 are designed to removably receive the stand elements 104 to elevate the jig assembly off a surface during film application. When not in use, the stand elements 104 can be secured in storage positions adjacent to the receptacles 106 by retention mechanisms 110.

The retention mechanisms 110 on the jig assembly 102 may include various integrated fastening approaches for securing the stand elements 104. For example, spring-loaded clips can be incorporated into the bottom surface of the jig assembly to provide positive retention force on the stand elements. Push-pin fasteners with detents can engage corresponding features machined into the stand elements, allowing for secure attachment and quick release when needed. The assembly may alternatively utilize friction-fit retaining clips that the stand elements can snap into for storage.

Additional retention options may include quarter-turn fasteners that allow rapid securing and removal of the stand elements, or slide-and-lock channels with integrated detents positioned adjacent to the receptacles 106. Resilient tabs molded into the bottom surface can flex to permit insertion while maintaining consistent retention force on the stored stand elements.

In some examples, these retention mechanisms 110 may be positioned on the bottom side of the jig assembly 102 near the cylindrical receptacles 106 to secure the stand elements 104 when they are not deployed for elevating the assembly. The retention system works in conjunction with the storage positions adjacent to the receptacles to create an organized and secure storage solution for the stand elements when not in use.

The jig assembly 102 may include provisions 108 along its peripheral edge that include slots designed to accommodate and engage the eyewear temples. These provisions 108 match the contour of the eyewear to maintain proper positioning during film application.

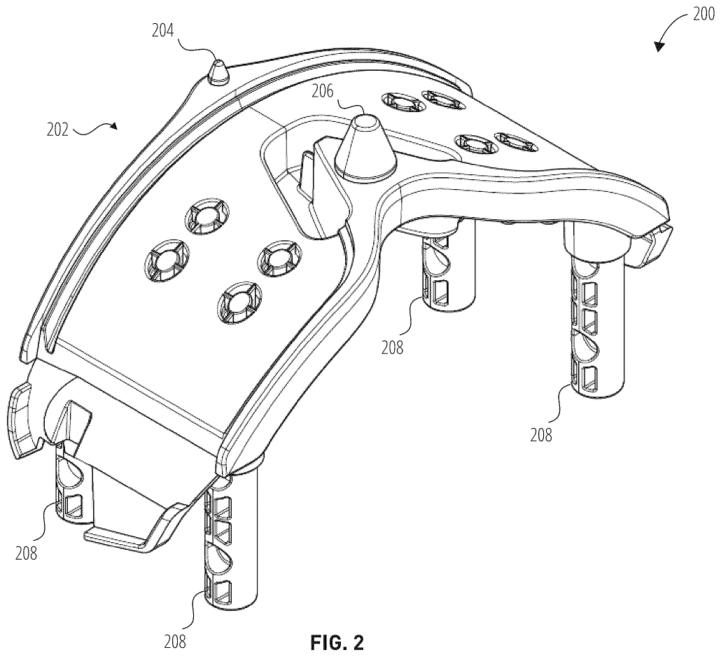

FIG. 2 is a diagram 200 of a jig assembly 202, in accordance with some examples, for positioning and application of protective films to eyewear. As seen in the diagram 200, the jig assembly 202 includes positioning pins 204, 206 arranged along a vertical axis on its front face, which are configured to align with corresponding features on the protective film to ensure proper placement during application. These positioning pins 204 and 206 may be implemented using various common configurations known to those skilled in the art, such as tapered guide pins for facilitating initial alignment, or pins with chamfered tips to aid in film alignment.

The jig assembly 202 includes multiple stand elements 208 that can be deployed to elevate the jig assembly 202 during film application. In some examples, the stand elements 208 are configured to be removably inserted into corresponding cylindrical receptacles formed in the bottom side of the jig assembly 202. For example, the receptacles can incorporate various retention features such as friction-fit interfaces, threaded connections for secure engagement, or quick-release mechanisms with detents for positive positioning.

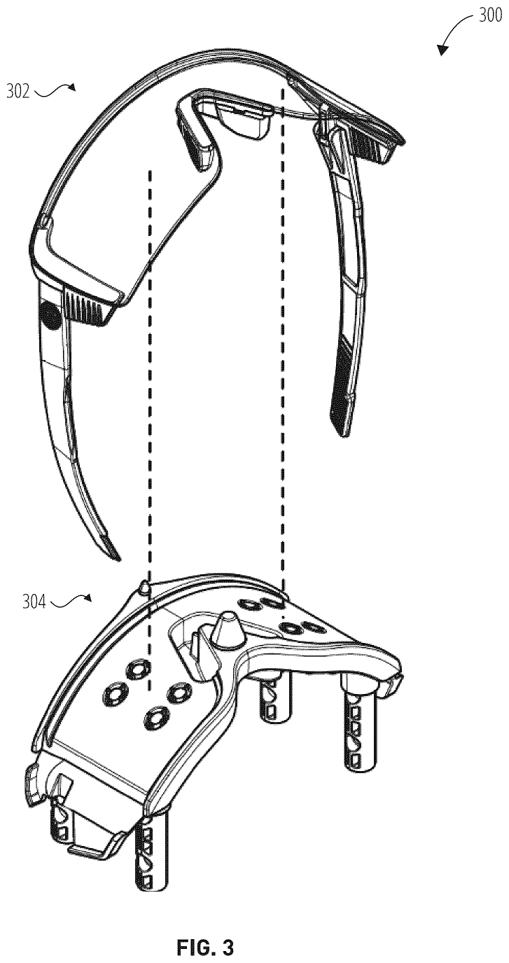

FIG. 3 is a diagram 300 depicting the positioning of eyewear 302 onto a jig assembly 304, according to certain examples. As seen in the diagram 300, the eyewear 302 includes temples extending from opposite sides of the lens, which are designed to engage with corresponding provisions formed along the peripheral edge of the jig assembly 304. These provisions include specifically configured slots that match the contour of the eyewear and accommodate the temples to ensure proper alignment and secure positioning.

In some examples, the jig assembly 304 features an alignment rim along its peripheral edge that has a contour specifically matched to the shape of the eyewear 302. When positioning the eyewear 302, the user aligns the temples with the dedicated slots in the alignment rim, allowing the eyewear to be lowered into a precise position on the jig assembly. The non-abrasive surface of the jig assembly 304 conforms to the curvature of the lens while preventing scratches during positioning.

The alignment process ensures that the eyewear 302 is held in a fixed position relative to the jig assembly 304 for subsequent film application. The temple engagement with the alignment rim's slots creates a secure fit that maintains the eyewear's position throughout the application process. Once the eyewear 302 is properly positioned on the jig assembly 304, the temples of the eyewear 302 may be folded inward and downward below the jig assembly 304. This configuration creates a compact assembly that serves both application and storage purposes.

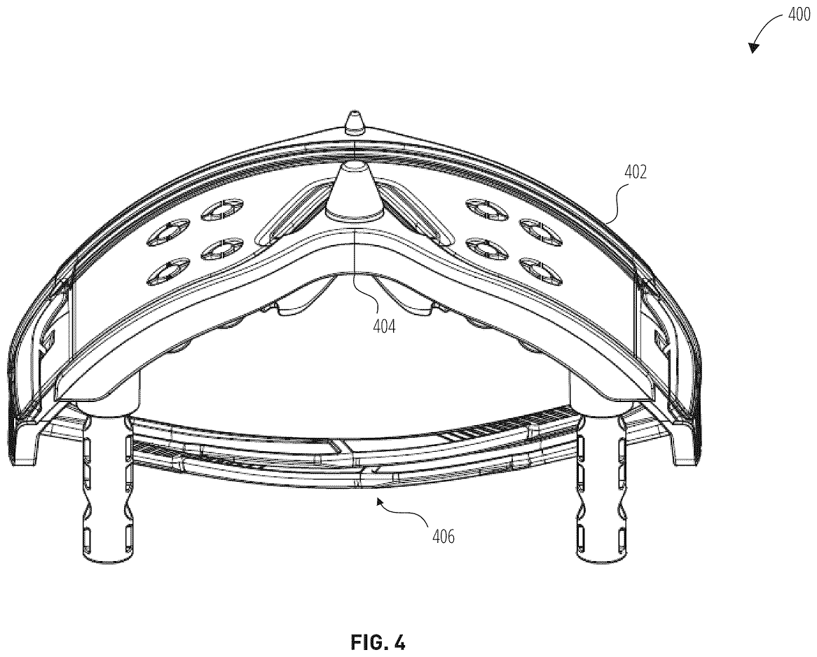

FIG. 4 is a diagram 400 depicting eyewear 402 secured in position on a jig assembly 404, in accordance with one embodiment. As seen in the diagram 400, the eyewear 402 is positioned with its lens surface conforming to the non-abrasive surface of the jig assembly 404, which is contoured to match the shape of the eyewear. The temples 406 of the eyewear 402 are shown folded inward and downward below the jig assembly 404, creating a compact and secure configuration.

The diagram 400 demonstrates how the eyewear 402 and jig assembly 404 form a cohesive unit once fully assembled. The folded position of the temples 406 helps maintain the eyewear's position during film application while also providing a space-efficient storage solution. The jig assembly 404 includes provisions along its peripheral edge that have guided the proper positioning of the eyewear 402, ensuring precise alignment for subsequent film application steps.

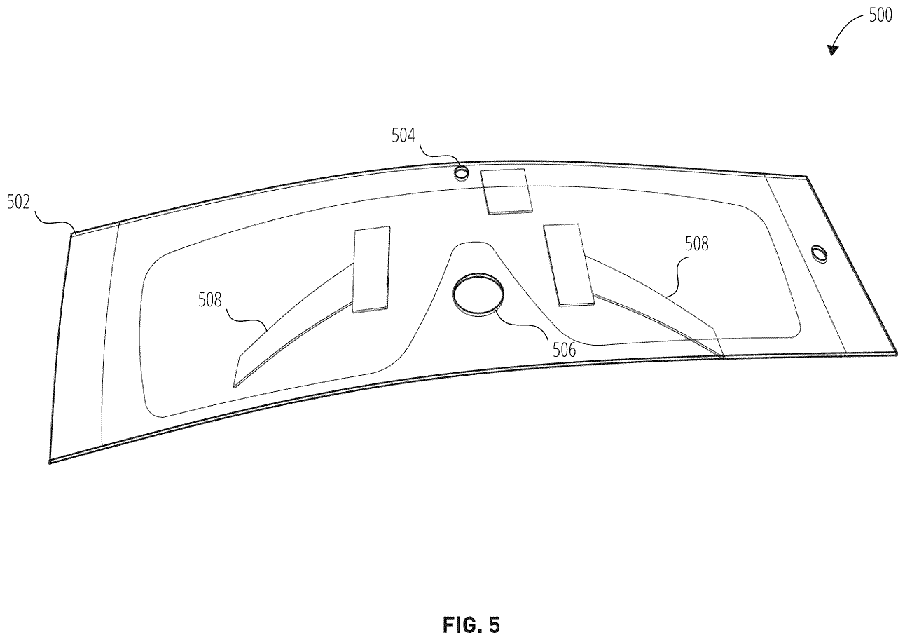

FIG. 5 is a diagram 500 depicting a protective film 502, in accordance with some examples. The protective film 502 includes alignment provisions 504, 506 that correspond with positioning pins on the jig assembly. While the alignment provisions may be arranged along a common vertical axis as shown in the diagram 500, they can alternatively be positioned in various other configurations that match the corresponding arrangement of positioning pins on the jig assembly. These alignment provisions ensure proper positioning of the film during application regardless of their specific arrangement on the film.

In some examples, the protective film 502 incorporates a segmented release liner system with independently removable sections. The film includes removal tabs 508 attached to the inner edges of the side sections of the release liner. These tabs 508 facilitate controlled removal of the release liner sections during the application process. The center section of the release liner corresponds with the alignment provisions 504, 506, allowing initial positioning of the film using the jig assembly's positioning pins before the side sections are removed.

The film 502 may be designed with an adhesive backing that provides secure attachment to the lens surface while allowing clean removal when needed. The segmented design of the release liner enables a systematic application process where the center section is removed first for initial alignment, followed by sequential removal of the side sections using the removal tabs 508. This staged approach helps prevent air bubbles and ensures proper adhesion to the curved lens surface.

The protective film 502 may be provided in various configurations, including colored or tinted versions that can be combined through stacking to achieve different visual effects. The film's design specifically accommodates lenses that curve along a single axis, allowing for proper adhesion during use and clean removal when the lens is bent in a direction opposite to its natural curvature.

In some examples, the protective film 502 may extend slightly beyond the lens area onto a portion of the frame or reinforcement rib. This extension creates a small tab or overhang that remains unattached to the curved lens surface, providing users with an easily accessible grip point for initiating film removal. Accordingly, when removal of the film is desired, users can grasp this extended portion of the film and pull gently to begin the separation process.

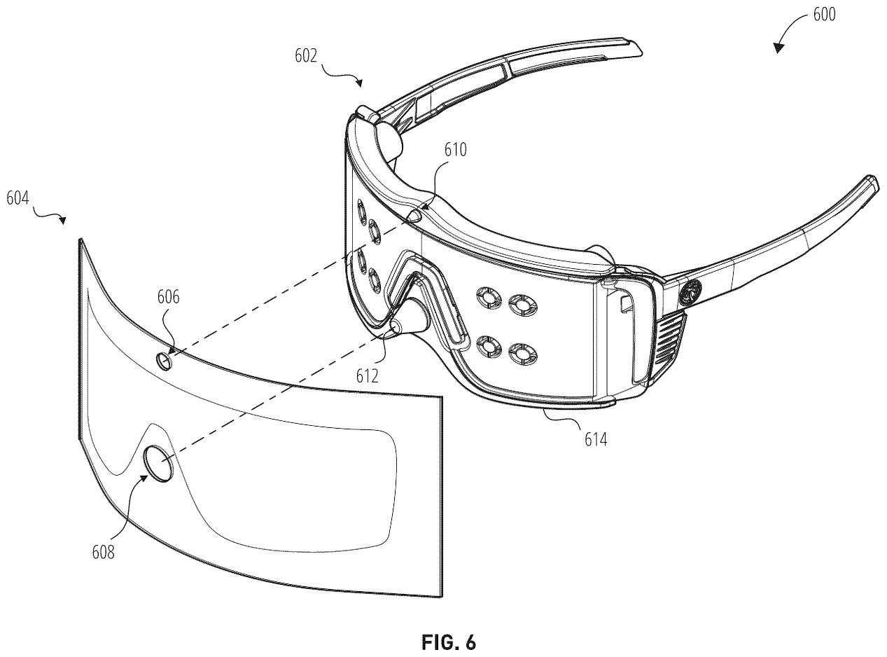

FIG. 6 is a diagram 600 depicting a protective film 604 being applied to eyewear 602, in accordance with some examples. As seen in the diagram 600, the protective film 604 includes alignment provisions 606, 608 that correspond with and engage positioning pins 610, 612 on the jig assembly 614. This alignment system ensures precise positioning of the film during the application process.

In some examples, the application process begins with the eyewear 602 secured in the jig assembly. While the temples may be folded beneath the assembly as one configuration, they can alternatively remain unfolded during the application process. The protective film 604 incorporates a segmented release liner system with independently removable sections. During application, a center section of the release liner is first removed to expose the adhesive backing in the area containing the alignment provisions 606, 608.

The film 604 is then positioned by engaging the alignment provisions 606, 608 with their corresponding positioning pins 610, 612. Once the initial alignment is achieved through the center section, the remaining side sections of the release liner are sequentially removed using dedicated removal tabs located along their inner edges. This staged removal process helps prevent air bubbles and ensures proper adhesion to the curved lens surface.

In some examples, the film 604 can be provided in various configurations, including colored or tinted versions that can be combined through stacking to achieve different visual effects.

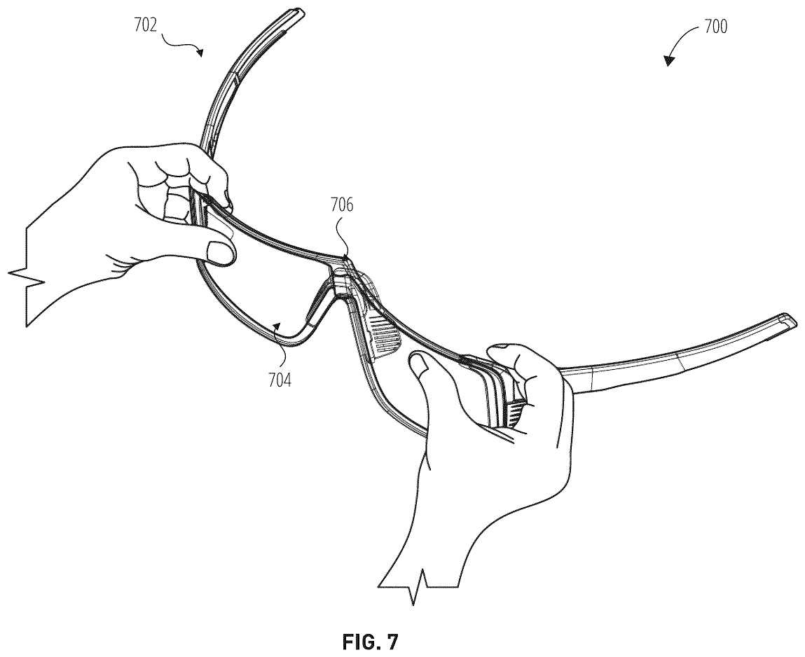

FIG. 7 is a diagram 700 depicting removal of a protective film 704 from eyewear 702, in accordance with some examples. The removal process may leverage the single-axis curvature of the lens by bending the eyewear 702 in a direction opposite to its natural curvature. This opposing force causes the protective film 704 to lift and separate from the lens surface in the central region 706, creating an accessible edge that allows for complete removal of the film.

When the lens of the eyewear 702 is bent counter to its natural curve, the differential in surface tension between the curved lens surface and the film's adhesive backing causes the film to naturally separate from the lens. This separation begins in the central region 706 of the lens, providing a natural lifting point for complete film removal.

### EXAMPLES

Example 1: A system comprises an eyewear that comprises a lens, a film to be applied to the lens of the eyewear, the film comprising an adhesive backing, a release liner, and one or more alignment features, and a jig assembly that comprises one or more positioning pins that corresponds with the one or more alignment features of the film, the jig assembly configured to retain the lens in a fixed position.

Example 2: The subject matter of Example 1, wherein the one or more positioning pins of the jig assembly comprises at least a first positioning pin and a second positioning pin.

Example 3: The subject matter of Example 1 or 2, wherein the jig assembly comprises a front face, and wherein the first positioning pin and the second positioning pin are positioned on the front face along a common vertical axis.

Example 4: The subject matter of any of Examples 1-3, wherein at least one positioning pin from among the one or more positioning pins comprise a tapered shape.

Example 5: The subject matter of any of Examples 1-4, wherein the one or more alignment features of the film comprise at least a first alignment feature and a second alignment feature.

Example 6: The subject matter of Example 5, wherein the film comprises a central segment, and wherein the first alignment feature and the second alignment feature are positioned upon the central segment along a common vertical axis that corresponds with a location of the one or more positioning pins on the jig assembly.

Example 7: The subject matter of any of Examples 1-6, wherein the release liner of the film comprises a plurality of release sections that are independently removable.

Example 8: The subject matter of any of Examples 1-7, wherein the eyewear comprises a first temple and a second temple, each extending from opposite sides of the eyewear, and the jig assembly further comprises an alignment rim along a peripheral edge of the jig assembly, wherein the alignment rim comprises a contour that matches a shape of the eyewear.

Example 9: The subject matter of any of Examples 1-8, wherein the jig assembly further comprises at least one stand element configured to elevate the jig assembly off a surface.

Example 10: The subject matter of Example 9, wherein the jig assembly further comprises a plurality of receptacles formed in a bottom side of the jig assembly, wherein the at least one stand element is configured to be removably inserted into a corresponding receptacle to elevate the jig assembly off the surface.

Example 11: The subject matter of Example 9 or 10, wherein the jig assembly further comprises one or more securing elements to releasably engage and hold the at least one stand element when the at least one stand element is not being used to elevate the jig assembly.

Example 12: A method comprises positioning eyewear that comprises a lens onto a jig assembly that comprises one or more positioning pins, aligning a film that comprises an adhesive backing and one or more alignment features with the one or more positioning pins of the jig assembly, and applying the film to the lens of the eyewear while the eyewear is retained in the jig assembly.

Example 13: The subject matter of Example 12, wherein the lens comprises a curvature, and the method further comprises removing the film by bending the lens in a direction opposite the curvature.

Example 14: The subject matter of Example 13, wherein the curvature is along a single axis.

Example 15: The subject matter of any of Examples 12-14, wherein the film comprises a release liner having a plurality of release sections that are independently removable.

Example 16: The subject matter of Example 15, wherein applying the film comprises removing a center section of the release liner, aligning the film with the positioning pins, and removing side sections of the release liner.

Example 17: The subject matter of any of Examples 12-16, wherein the jig assembly comprises an alignment rim along a peripheral edge, the alignment rim having a contour that matches a shape of the eyewear.

Example 18: The subject matter of Example 17, wherein positioning the eyewear comprises engaging temples of the eyewear with slots in the alignment rim.

Example 19: The subject matter of any of Examples 12-18, wherein the jig assembly comprises at least one stand element configured to elevate the jig assembly off a surface during the applying of the film.

Example 20: The subject matter of any of Examples 12-19, wherein the one or more positioning pins of the jig assembly comprises at least a first positioning pin and a second positioning pin positioned along a common vertical axis.

Example 21: An apparatus comprising means to perform one or more elements of a method described in or related to any of Examples 1-20 or any other method or process described herein.

Example 24: A method, technique, or process as described in or related to any of Examples 1-23 or portions or parts thereof.

What is claimed is:

1. A system comprising:
an eyewear that comprises a lens;
a film to be applied to the lens of the eyewear, the film comprising an adhesive backing, a release liner, and one or more alignment features; and
a jig assembly that comprises one or more positioning pins that corresponds with the one or more alignment features of the film, the jig assembly configured to retain the lens in a fixed position.

2. The system of claim 1, wherein the one or more positioning pins of the jig assembly comprises at least a first positioning pin and a second positioning pin.

3. The system of claim 2, wherein the jig assembly comprises a front face, and wherein the first positioning pin and the second positioning pin are positioned on the front face along a common vertical axis.

4. The system of claim 1, wherein at least one positioning pin from among the one or more positioning pins comprise a tapered shape.

5. The system of claim 1, wherein the one or more alignment features of the film comprise at least a first alignment feature and a second alignment feature.

6. The system of claim 5, wherein the film comprises a central segment, and wherein the first alignment feature and the second alignment feature are positioned upon the central segment along a common vertical axis that corresponds with a location of the one or more positioning pins on the jig assembly.

7. The system of claim 1, wherein the release liner of the film comprises a plurality of release sections that are independently removable.

8. The system of claim 1, wherein the eyewear comprises a first temple and a second temple, each extending from opposite sides of the eyewear, and the jig assembly further comprises an alignment rim along a peripheral edge of the jig assembly, wherein the alignment rim comprises a contour that matches a shape of the eyewear.

9. The system of claim 1, wherein the jig assembly further comprises at least one stand element configured to elevate the jig assembly off a surface.

10. The system of claim 9, wherein the jig assembly further comprises a plurality of receptacles formed in a bottom side of the jig assembly, wherein the at least one stand element is configured to be removably inserted into a corresponding receptacle to elevate the jig assembly off the surface.

11. The system of claim 9, wherein the jig assembly further comprises one or more securing elements to releasably engage and hold the at least one stand element when the at least one stand element is not being used to elevate the jig assembly.

12. A method comprising:
positioning eyewear that comprises a lens onto a jig assembly that comprises one or more positioning pins;
aligning a film that comprises an adhesive backing and one or more alignment features with the one or more positioning pins of the jig assembly; and
applying the film to the lens of the eyewear while the eyewear is retained in the jig assembly.

13. The method of claim 12, wherein the lens comprises a curvature, and the method further comprises:
removing the film by bending the lens in a direction opposite the curvature.

14. The method of claim 13, wherein the curvature is along a single axis.

15. The method of claim 12, wherein the film comprises a release liner having a plurality of release sections that are independently removable.

16. The method of claim 15, wherein applying the film comprises:
removing a center section of the release liner;
aligning the film with the positioning pins; and
removing side sections of the release liner.

17. The method of claim 12, wherein the jig assembly comprises an alignment rim along a peripheral edge, the alignment rim having a contour that matches a shape of the eyewear.

18. The method of claim 17, wherein positioning the eyewear comprises engaging temples of the eyewear with slots in the alignment rim.

19. The method of claim 12, wherein the jig assembly comprises at least one stand element configured to elevate the jig assembly off a surface during the applying of the film.

20. The method of claim 12, wherein the one or more positioning pins of the jig assembly comprises at least a first positioning pin and a second positioning pin positioned along a common vertical axis.
